(19) United States
(12) Patent Application Publication
(10) Pub. No.: US 2013/0296390 A1
(43) Pub. Date: Nov. 7, 2013

(54) METHOD AND COMPOSITIONS FOR ENHANCING THE SAFETY OF ORALLY ADMINISTERED MAGNESIUM ALPHA-LIPOATE

(71) Applicant: BIOLINK LIFE SCIENCES, INC., Cary, NC (US)

(72) Inventor: Deanna J. Nelson, Raleigh, NC (US)

(73) Assignee: BioLink Life Sciences, Inc., Cary, NC (US)

(21) Appl. No.: 13/873,677

(22) Filed: Apr. 30, 2013

Related U.S. Application Data
(60) Provisional application No. 61/643,660, filed on May 7, 2012.

(51) Int. Cl. A61K 31/4188; A61K 31/381

(52) U.S. Cl. CPC A61K 31/4188; A61K 31/381
USPC 514/393

(57) ABSTRACT

The present invention relates to oral nutritional and therapeutic products which are useful for enhancing the safety of administering and reducing the adverse effects caused by magnesium alpha-lipoate to a warm-blooded mammal, comprising administering magnesium alpha-lipoate with D-biotin.

### FIELD OF THE INVENTION

The present invention relates to methods and compositions useful for enhancing the safety of orally administered magnesium alpha-lipoate. The methods and compositions of the present invention are particularly useful in mammals.

### BACKGROUND OF THE INVENTION

In reflection of its remarkable physiological properties, alpha-lipoic acid (ALA, general formula 1, X═OH) is one of today's most commonly used dietary supplements. (Other common names for ALA include lipoic acid, thiooctic acid, 1,2-dithiolane-3-pentanoic acid, 1,2-dithiolane-3-valeric acid, or 6,8-thiooctic acid.) ALA and its reduced form, dihydrolipoic acid (DHLA, general formula 2, X═OH), constitute a “universal” redox couple that is present as a protein-linked constituent of many biological systems in plants, humans, animals, and various microorganisms.

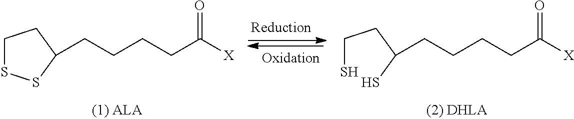

ALA has generated extensive interest as a “naturally occurring” anti-oxidant having the capacity to neutralize a variety of reactive oxygen, nitrogen, and sulfur species associated with disease and aging, as well as restore the biological activity of other physiological antioxidants such as glutathione, vitamin C, and vitamin E. One consequence of these beneficial properties has been the commercial availability and widespread use of ALA as a dietary supplement since the 1950's.

Concurrently, pharmaceutical therapies containing alpha-lipoic acid are being developed in pharmaceutical, academic, and research laboratories. The differing pharmacological properties of the individual enantiomers and racemates of ALA have become particularly significant with the resurgence of interest in ALA as a therapeutic agent and/or adjuvant treatment for major chronic diseases including diabetes, cardiovascular diseases, chronic kidney disease, hypertension, metabolic syndrome, and hyperlipidemia. The potential importance of ALA in mitigating the effects of or treating these widespread “killer” diseases is reflected in two facts: (1) These diseases accounted for almost 50% of the $500 billion in direct costs that the United States spent on health care. (2) The risk and prevalence of these disorders is increasing, suggesting that costs associated with them will continue both to increase and account for almost half of direct health care costs both today and in the future.

What structural formula of ALA shown above does not make clear is that ALA is a chiral compound having two, non-superimposable stereoisomers (enantiomers; structural formulae below, Chemical Abstracts Registry Numbers shown parenthetically). Mixtures of the two enantiomers constitute racemic ALA, the form that is most widely used as a dietary supplement.

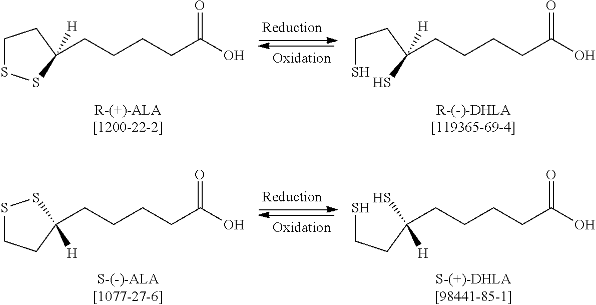 [SMILES: O=C(O)CCCC[C@H]1CCSS1 ; O=C(O)CCCC[C@H](S)CCS ; O=C(O)CCCC[C@H]1CCSS1 ; O=C(O)CCCC[C@H](S)CCS]

A review of the published literature indicates that a human may ingest oral doses of ALA in total doses ranging from 100 mg to as high as 1800 mg daily for months or years without overt adverse effects. Likewise, ALA has been administered orally to animals in chronic daily doses ranging from about 30 mg/kg body weight to as high as over 2,000 mg/kg body weight without overt adverse effects. [Shay K P, Moreau R F, Smith E J, Smith A R, Hagen T M. Alpha-lipoic acid as a dietary supplement: Molecular mechanisms and therapeutic potential. Biochim Biophys Acta 2009; 1790(10): 1149-1160.]

Therefore, the adverse findings of hair loss and skin disorders revealed in a recent study of the efficacy and safety of magnesium R-(+)-alpha lipoate administered orally to rodents were both unexpected and a significant deterrent to further development of magnesium alpha-lipoate as a nutrient and treatment for chronic diseases in humans and animals. The present invention provides a solution for mitigating the adverse effects associated with administration of magnesium alpha-lipoate and meets the significant unmet need for enhancing the safety of magnesium alpha-lipoate for use as a nutrient and treatment for chronic diseases in humans and animals.

### SUMMARY OF THE INVENTION

The present invention comprises oral nutritional and therapeutic compositions useful for enhancing the safety of magnesium alpha-lipoate, comprising a unit dosage or serving of magnesium alpha-lipoate with D-biotin. Methods of enhancing the safety of administration of magnesium alpha-lipoate in a human, comprising administering to said human a safe and effective amount of a supplement comprising magnesium alpha-lipoate with D-biotin, are also disclosed. Further, a method of enhancing the safety of magnesium alpha-lipoate administration in a warm-blooded animal, comprising administering a therapeutically effective amount of a pharmaceutical composition comprising magnesium alpha-lipoate with D-biotin is disclosed. Other features, advantages, and embodiments of the invention will be apparent to those of ordinary skill in the art from the following description, examples, and appended claims.

### DESCRIPTION OF THE DRAWINGS

FIG. 1 is a photograph showing the overt adverse effects of administering a chronic daily dose of 0.5% or 1.5% (by weight) of magnesium R-(+)-alpha-lipoate in the diet of a LDLR−/−mouse. Animals receiving magnesium R-(+)-alpha-lipoate exhibited hair loss and skin disorders.

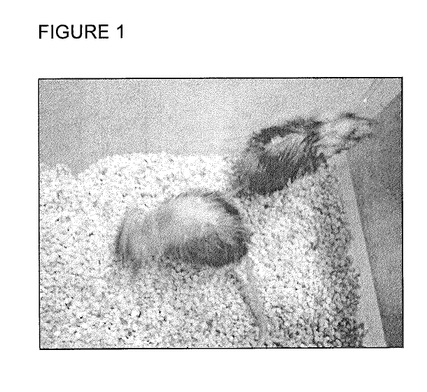

FIG. 2 is a photograph showing the absence of overt adverse effects when a chronic daily dose of 0.5% or 1.5% (by weight) of magnesium R-(+)-alpha-lipoate with D-biotin is administered in the diet of a LDLR−/−mouse.

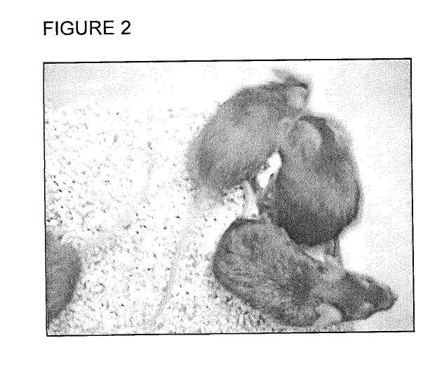

### DETAILED DESCRIPTION OF THE INVENTION

The present invention is an oral nutritional and therapeutic composition useful for enhancing the safety of administration of magnesium alpha-lipoate, comprising a unit dosage or serving of magnesium alpha-lipoate with D-biotin. The composition is useful in mammals.

The present invention also relates to a method of enhancing the safety of ingestion of magnesium alpha-lipoate in a human, comprising administering to said human a safe and effective amount of a nutrient supplement comprising effective amounts of magnesium alpha-lipoate with D-biotin.

In addition, the present invention relates to a method of enhancing the safety of therapeutically effective amounts of a pharmaceutical composition comprising magnesium alpha-lipoate when administered to a warm-blooded animal, comprising administering to said warm-blooded animal safe and effective amounts of magnesium alpha-lipoate and D-biotin. Included within the scope of this invention is a method of enhancing the safety of therapeutically effective amounts of a pharmaceutical composition comprising magnesium alpha-lipoate when administered orally to a warm-blooded animal, comprising administering pharmaceutical compositions comprising magnesium R-(+)-alpha-lipoate with D-biotin and a suitable pharmaceutical carrier.

The term “magnesium lipoate” refers to the magnesium salt of alpha-lipoic acid. The term refers to the magnesium salt of a single enantiomer of alpha-lipoate or the magnesium salt of a racemic mixture of a-lipoate (i.e., magnesium (R)- or (S)-alpha-lipoate or magnesium (RS)-alpha-lipoate, respectively). Magnesium a-lipoate is a stable, non-hygroscopic, light yellow powder having a molecular formula of Mg(C₈H₁₃O₂S₂)₂, the general formula

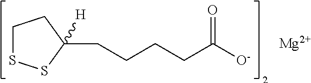

and a molecular weight of 434.94. Magnesium R-(+)-alpha-lipoate is the magnesium salt of R-(+)-α-lipoic acid. Magnesium R-(+)-α-lipoate is a stable, non-hygroscopic, light yellow powder having a molecular formula of Mg(C₈H₁₃O₂S₂)₂, the general formula

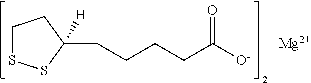

and a molecular weight of 434.94.

The term “biotin” means D-biotin, an essential water-soluble vitamin also known as Vitamin H, Coenzyme R, or vitamin B7. Biotin has Chemical Abstracts Service Registry No. 58-85-5 and the general formula:

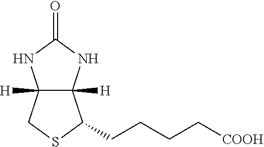

Biotin is a white amorphous powder having the molecular formula C₁₀H₁₆N₂O₃S and a molecular weight of 244.31 g/mol. Also within the scope of this term are physiologically compatible salts of biotin, hydrates, crystalline forms, polymorphic forms, solid forms having specific bulk densities or tap densities, and solid forms having specific particle sizes. Further included within the scope of this term are biotin compositions coated with pharmaceutically acceptable materials intended to modify its release and/or bioavailability (e.g., Eudragit, microcrystalline cellulose, hydroxypropylmethylcellulose phthalate, and so forth).

Biotin is specifically taken up from the diet by intestinal sodium-dependent vitamin transporters (SMVT) and is non-specifically transported by monocarboxylate transporters in the intestine. These same transporters also mediate intracellular transport of the vitamin. For example, in keratinocytes, the SMVT transport system exhibited a Michaelis-Menten constant for biotin of 22.7±1.0 μM and a maximal velocity of 163.6±3.5 pmol per five minutes per milligram of protein. Biotin uptake was strongly inhibited by ALA (Kᵢ=4.6 μM), dihydrolipoic acid (the reduced dithiol form of ALA; Kᵢ=11.4 μM), panthothenic acid (Kᵢ=1.2 μM) and desthiobiotin (Kᵢ=15.2 μM) but not by biocytin or biotin methyl ester. [Grafe F, Wohlrab W, Neubert R H, Brandsch M. Transport of biotin in human keratinocytes. J Invest Dermatol 2003; 120: 428-433.} The second biotin transport component is saturable at very low biotin concentrations (Kᵢ=2.6±0.1 nM) but not inhibited by ALA and pantothenic acid. In addition, endogenous reutilization of biotin and capture of biotin generated in intestinal flora serve to maintain adequate biotin to maintain nutritional requirements.

Biotin serves as a coenzyme for five carboxylases that catalyze pathways involved in fatty acid biosynthesis, gluconeogenesis, branched-chain amino and fatty acid metabolism, tricarboxylic acid cycle anaplerosis, and pleiotropic gene regulation, particularly for genes in carbohydrate metabolism. In addition to its critical role as a prosthetic cofactor for enzymes that catalyze carboxylation, biotin plays a significant role in cell proliferation and differentiation and histone activity [Zempleni J, Hassan Y I, Wijeratne S S K. Biotin and biotinidase deficiency. Expert Rev Endocrinol Metab 2008 Nov. 1; 3(6): 715-724.].

The term “magnesium” means the magnesium ion, Mg²⁺.

The term “bioavailability” refers to the amount of a substance that is absorbed in the intestines and ultimately available for biological activity in a subject's cells and tissues.

The term “excipient material” is intended to mean any compound forming a part of the formulation which is not intended to have biological activity itself and which is added to a formulation to provide specific characteristics to the dosage form, including by way of example, providing protection to the active ingredient from chemical degradation, facilitating release of a tablet or caplet from equipment in which it is formed, and so forth.

The term “enhancing the safety” and the like are used herein to generally mean obtaining a desired pharmacological and physiological effect of reducing the incidence, risk, or severity of at least one adverse side effect associated with administration of a composition to a mammal. The effect may be prophylactic in terms of preventing or partially preventing the incidence, risk, or severity of an adverse symptom or condition caused by or related to the administration of a therapeutic agent.

The terms “preventing”, “treating”, “treatment” and the like are used herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein encompasses any treatment of a disease in a mammal, particularly a human and includes: (a) preventing the disease from occurring in a subject which may be predisposed to the disease but has not yet been diagnosed as having it; (b) inhibiting the disease or arresting its development; or (c) relieving the disease, causing regression of the disease and/or its symptoms, conditions, and co-morbidities.

The phrase “therapeutically effective” is intended to qualify the amounts of magnesium alpha-lipoate and biotin which will achieve the goal of providing the quantity of biotin needed to prevent and treat adverse effects associated with the ingestion of magnesium alpha-lipoate. The amounts of magnesium alpha-lipoate and D-biotin may be administered orally as part of the same unit dose or as different unit doses administered in a coordinated manner that supplies both magnesium alpha-lipoate and biotin to the subject. Further, the amounts of magnesium alpha-lipoate and D-biotin may be administered in a coordinated manner by different routes of administration, if required to ensure bioavailability in a subject requiring this treatment. By way of example, administration in a coordinated manner may comprise oral administration of an effective amount of magnesium alpha-lipoate at a time point and administration of an effective amount of D-biotin by oral or intravenous administration at a separate time point within 72 hours of administration of magnesium alpha-lipoate.

For the purpose of this disclosure, a warm-blooded animal is a member of the animal kingdom which includes but is not limited to mammals and birds. The most preferred mammal of this invention is human.

To provide a more concise description, some of the quantitative expressions given herein are not qualified with the term “about.” It is understood that whether the term “about” is used explicitly or not, every quantity give herein is meant to refer to the actual given value, and it is also meant to refer to the approximation to such given value that would reasonably be inferred based on the ordinary skill in the art, including approximations due to the experimental and/or measurement conditions for such given value.

The inventor recently completed an animal study that critically examined the in vivo efficacy of phosphate binding by a combination of phosphate binders, magnesium R-(+)-α-lipoate and calcium succinate monohydrate. (Details of the study are provided in Example 1.) A mouse model of chronic kidney disease (CKD), partial renal ablation in the LDL receptor-deficient (LDLR−/−) mouse that is fed high fat/cholesterol diets, was used. This model resembles the clinical situation of CKD complicated by the metabolic syndrome, because the mice exhibit obesity, hypertension, insulin resistance, and early type II diabetes. In these animals, CKD causes intensification of vascular calcification (VC). The inventor expected to observe significant binding of dietary phosphate in the intestines of test animals, and the experimental data confirmed that significant phosphate binding occurred. However, unexpectedly, test animals which received a diet composed of 0.5% calcium succinate/0.5% magnesium lipoate or 1.5% calcium succinate/1.5% magnesium lipoate (by weight) in rodent chow exhibited extensive hair loss and skin disorders (e.g., pruritis). Control animals receiving the same rodent chow diet did not show these adverse effects. The overt adverse effects observed in the test animals were sufficiently serious to stop the study and halt continuing development of magnesium alpha-lipoate as a dietary supplement and therapeutic treatment for humans and animals. In order to continue the study and to complete commercial development of the calcium succinate/magnesium lipoate test formulation, the adverse effects had to be eliminated.

A review of the published literature provided the following facts regarding the test compositions of Example 1. The rodent chow formulation did not cause hair loss or skin disorders. Neither calcium nor magnesium is known to cause hair loss or skin disorders in the concentrations administered. Succinate is a physiological intermediate in the tricarboxylic acid cycle. Exogenously administered succinate is not known to cause hair loss or skin disorders in the concentrations administered.

Alpha-Lipoic acid is the sole remaining component in the formulation tested in Example 1. The safety and absence of toxicity of ALA has been established in both short and long-term toxicity studies, including in vitro mutagenicity/genotoxicity studies. The no-observed adverse effect level (NOAEL) of ALA is considered to be 60 mg per kg body weight per day. (For an adult man this corresponds to 4.2 grams of ALA per day, and for an adult woman this is equivalent to 3 grams of ALA per day.) Indeed, published studies of alpha-lipoate, in quantities of 100 to about 1800 mg lipoate administered by mouth to animals or humans daily, do not report the adverse events (i.e., hair loss, skin disorders, etc.) seen in the testing performed by the inventor (Example 1). Nonetheless, the inventor examined to a number of potential mechanisms of toxicity related to alpha-lipoate.

For example, the inventor hypothesized that exogenously administered lipoate is reduced intracellularly by several enzymes and released into the extracellular milieu as dihydrolipoate. Dihydrolipoate chelates transition metals in biological systems. Chelation may alter the solubility and membrane permeability of the metal and may, in fact, mobilize toxic metals which may be adventitiously present in the diet. A non-physiological selenium burden, for example, would cause hair loss. However, the absence of similar reports from published studies of orally administered lipoate in both animals and humans suggests that this hypothetical rationale for the toxicities observed by the inventor has no scientific validity.

Alternatively, the inventor hypothesized that competitive inhibition of zinc uptake from the GI tract by calcium and magnesium as well as copper or zinc chelation by the sulfur atoms of dihydrolipoate in the intestinal lumen may prevent uptake of adequate copper and/or zinc from the diet. (In vitro studies show that LA preferentially binds to Cu²⁺, Zn²⁺, Pb²⁺, Hg²⁺, and Fe³⁺. [Shay K P, Moreau R F, Smith E J, Smith A R, Hagen T M. Alpha-lipoic acid as a dietary supplement: Molecular mechanisms and therapeutic potential. Biochim Biophys Acta 2009; 1790(10): 1149-1160.] However, the absence of similar reports from published studies of orally administered lipoate in both animals and humans suggests that this hypothetical rationale for the toxicities observed by the inventor has no scientific validity.

In the alternative, the inventor examined the hypothetical rationale for the toxicities based on the fact that a-lipoate undergoes predominantly beta-oxidation to a variety of analogs with shortened carbon side chains. Each metabolite undergoes intracellular reduction in the same manner as ALA itself. Both dihydrolipoate and its dimercapto-carboxylic acid metabolites can be further metabolized to the corresponding S-bismethylated carboxylic acids in reactions catalyzed by endogenous S-methyl transferases, reactions that apparently up-regulated in renally impaired subjects. Furthermore, there is evidence of glucuronic acid conjugation of ALA and some of its metabolites, as well as glycine conjugation in mice. Intravenous administration of an acute 100 mg/kg dose of ALA in a rat model induced elevated S-adenosylhomocysteine and depleted S-adenosylmethionine. [Stabler S P, Sekhar J, Allen R H, O'Neill H C, White C W. a-Lipoic acid induces elevated S-adenosylhomocysteine and depletes S-adenosylmethionine. Free Rad Biol Med 2009; 47: 1147-1153.] Thus, high concentrations of ALA in the body may present a methylation burden with severe depletion of methylating entities and accumulation of non-physiological sulfur amino acids, particularly in animals with compromised renal function. However, no significant toxicities have been observed in human studies involving both normal subjects and those with chronic kidney disease. [Teichert J, Preiss R. Pharmacokinetics, metabolism, and renal excretion of alpha-lipoic acid and its metabolites in humans. Chapter 11, pp 271-292, in: Patel M S, Packer L, Eds. Lipoic Acid: Energy Production, Antioxidant Activity and Health Effects. CRC Press, Boca Raton, 2008.] Therefore, the absence of similar reports from published studies of orally administered lipoate in both animals and humans suggests that this hypothetical rationale for the toxicities observed by the inventor has no scientific validity.

In the alternative, the inventor examined the hypothetical rationale for the observed toxicities based on the fact that lipoate interferes with biotin uptake or physiological activity. ALA is fat-soluble (i.e., lipid-soluble) and slightly water soluble. As a result, orally administered ALA is absorbed almost quantitatively into cells and the systemic circulation of the body (i.e., greater than 90%). Since most of the orally administered ALA is absorbed within two hours, the rapid bioavailability of ALA is attributed to absorption from the stomach. Recent studies have shown that orally administered ALA is also absorbed by enterocytes in the intestine. Enterocytic uptake of ALA in the intestine is mediated by a sodium-dependent multivitamin transporter (SMVT), a biological transporter that also transports biotin and pantothenic acid. Therefore, the inventor hypothesized that magnesium alpha-lipoate might interfere with ALA uptake. Published reports concerning oral administration of ALA to humans in total doses ranging from 100 mg to as high as 1800 mg daily for months or years found no overt adverse effects. Likewise, published reports confirm that ALA has been administered orally to animals in chronic daily doses ranging from about 30 mg/kg body weight to as high as over 2,000 mg/kg body weight without overt adverse effects. Thus, the prior art, which referenced no observations of hair loss or skin disorders after administration of ALA or its lipoate salts, provided no clinical or scientific support for this hypothetical rationale of the toxicities of alpha-lipoate observed by the inventor.

Likewise, it is known that ALA administered intravenously, reduces the activities of biotin-dependent carboxylases, namely, the mitochondrial β-methylcrotonyl-CoA carboxylase (EC 6.4.1.4), propionyl-CoA carboxylase (EC 6.4.1.3) and pyruvate carboxylase (EC 6.4.1.1) as well as cytosolic acetyl-CoA carboxylase (EC 6.4.1.2). These enzymes mediate key metabolic conversions of fatty acids and branched-chain amino acids. For example, Zemplini et al. found that intraperitoneal doses of ALA of 8.8 mg/kg·day or 31.8 mg/kg·d reduced the activities of pyruvate carboxylase and β-methylcrotonyl carboxylase by 28-36% in ALA-treated rats compared with vehicle controls (P<0.05) and decreased the activity of acetyl carboxylase by as much as 43% (with wide intragroup variability confounding a determination of significance). [Zempleni J, Trusty T A, Mock D M. Lipoic acid reduces the activities of biotin-dependent carboxylases in rat liver. J Nutr 1997; 127( ): 1776-1781.] However, the authors did not report hair loss or skin disorders as a result of intravenous ALA treatment. Thus, this hypothetical rationale for the toxicities observed by the inventor had no scientific or clinical support from the prior art.

Through completion of a second animal study (Example 2) under the same conditions as the first (Example 1), the inventor has surprisingly discovered that the addition of biotin to the diet of animals receiving oral magnesium lipoate prevents the adverse effects seen in the first study (e.g., hair loss, skin disorders, etc). In the second study, LDL receptor-deficient (LDLR−/−) mice that were fed high fat/cholesterol diets, were used. Test animals received a diet composed of 0.5% calcium succinate/0.5% magnesium lipoate plus 0.001% biotin in rodent chow (by weight). Experimental data confirmed that both calcium and magnesium from the test formulation prevented absorption of dietary phosphate. In addition, experimental data from the second study confirmed that the addition of biotin to the test formulation prevented hair loss and skin disorders (e.g., pruritis), adverse effects seen in the first animal study (Example 1).

Thus, the inventor has discovered that provision of biotin with a magnesium alpha-lipoate composition provides a biocompatible and physiologically useful composition which, when administered to animals or humans, causes no overt adverse effects such as hair loss and skin disorders. Further, the inventor has discovered that a method for eliminating the adverse effects and toxicities caused by magnesium alpha-lipoate, comprising the administration of magnesium alpha-lipoate with biotin. The amounts of magnesium alpha-lipoate and D-biotin may be administered orally as part of the same unit dose or as different unit doses administered in a coordinated manner that supplies both magnesium alpha-lipoate and biotin to the subject. Further, the amounts of magnesium alpha-lipoate and D-biotin may be administered in a coordinated manner by different routes of administration, if required to ensure bioavailability to a subject requiring this treatment. By way of example, administration in a coordinated manner may comprise oral administration of an effective amount of magnesium alpha-lipoate at a time point and administration of an effective amount of D-biotin by oral or intravenous administration at a separate time point within 72 hours of administration of magnesium alpha-lipoate.

A composition of the invention contains from 5 mg to about 100 mg magnesium, on an elemental basis, in the form of magnesium alpha-lipoate (i.e., magnesium (R,S)-alpha-lipoate), magnesium (R)-(+)-alpha-lipoate, or magnesium (S)-(−)-alpha-lipoate administered in a coordinated manner with from about 0.01 mg to about 50 mg D-biotin. A clinician has the training and expertise to determine which dose of each active ingredient and which route of administration is most appropriate for a patient.

While not wishing to be bound by any particular hypothesis or theory, the inventor believes that magnesium alpha-lipoate interferes with uptake of D-biotin from the gastrointestinal tract and its cellular metabolism in multiple ways. For example, magnesium alpha-lipoate may inhibit the activity of biotinidase, the enzyme that hydrolyzes the amide bond between biotin and the epsilon-amino group of lysine, thus rendering biotin available for uptake. Likewise, magnesium alpha-lipoate may provide sufficient lipoate to inhibit transport of D-biotin by the sodium-dependent multivitamin transporter (SMVT), as does alpha-lipoic acid. Unlike alpha-lipoic acid, magnesium alpha-lipoate may also exchange with D-biotin (a carboxylic acid) to form an insoluble magnesium biotinate salt which is not bioavailable. This process would prevent transport of D-biotin by the SMVT and would also prevent uptake of D-biotin by the monocarboxylate transporter (the second biotin transporter). Exchange between magnesium alpha-lipoate and D-biotin to form insoluble magnesium biotinate would also prevent reabsorption after D-biotin release by colonic bacteria. If so, the inventor's surprising discovery that magnesium alpha-lipoate causes hair loss, pustules, and skin disorders, and may also cause additional unknown adverse effects indicates that one of the adverse effects of administration of magnesium alpha-lipoate is a significant and unexpected physiological biotin deficiency. This invention provides a solution to this heretofore unrecognized problem.

The compositions of this invention can be administered by any means that effects contact of the active ingredients with the site of action in the body of a warm-blooded animal. A most preferred means of administration is by the oral route (i.e., ingestion). The compositions of this invention can be administered as a single unit dose or administered as different unit doses separately containing magnesium alpha-lipoate and D-biotin. The amounts of magnesium alpha-lipoate and D-biotin may be administered orally as part of the same unit dose or as different unit doses administered in a coordinated manner that supplies both magnesium alpha-lipoate and biotin to the subject. Further, the amounts of magnesium alpha-lipoate and D-biotin may be administered in a coordinated manner by different routes of administration, if required to ensure bioavailability in a subject requiring this treatment. By way of example, administration in a coordinated manner may comprise oral administration of an effective amount of magnesium alpha-lipoate at a time point and administration of an effective amount of D-biotin by oral or intravenous administration at a separate time point within 72 hours of administration of magnesium alpha-lipoate. Compositions of this invention can be administered one or more times each day, so as to facilitate and enhance compliance with dosage regimens.

The active ingredients (i.e., magnesium alpha-lipoate and D-biotin) can be administered by the oral route in solid dosage forms, such as tablets, capsules, and powders, or in liquid dosage forms, such as elixirs, syrups, and suspensions. Each active ingredient can be administered by the parenteral route in liquid dosage forms. The pharmaceutical composition is preferably made in the form of a dosage unit containing a particular amount of each active ingredient. One most preferred oral dosage form of a composition of the present invention is an admixture of powders contained within a sachet. Because a composition of the present invention is not hygroscopic and has no repugnant taste or odor, the admixture of powders comprising a composition of the present invention can be sprinkled on food or stirred into beverages to enhance ease of use and support high levels of compliance with daily dosage regimens.

In general, the pharmaceutical dosage forms of compositions of this invention can be prepared by conventional techniques, as are described in Remington's Pharmaceutical Sciences, a standard reference in this field [Gennaro A R, Ed. Remington: The Science and Practice of Pharmacy. 20^{th} Edition. Baltimore: Lippincott, Williams & Williams, 2000]. For therapeutic purposes, the active components of this combination therapy invention are ordinarily combined with one or more adjuvants appropriate to the indicated route of administration. If administered per os, the components may be admixed with lactose, sucrose, starch powder, cellulose esters of alkanoic acids, cellulose alkyl esters, talc, stearic acid, magnesium stearate, gelatin, acacia gum, sodium alginate, polyvinylpyrrolidone, and/or polyvinyl alcohol, and then tabletted or encapsulated for convenient administration. Such capsules or tablets may contain a controlled-release formulation as may be provided in a dispersion of active compound in hydroxypropyl methylcellulose. Solid dosage forms can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. Both the solid and liquid oral dosage forms can contain coloring and flavoring to increase patient acceptance. Other adjuvants and modes of administration are well and widely known in the pharmaceutical art.

Dosing for oral administration may be with a regimen calling for single daily dose, or for a single dose every other day, or for a single dose within 72 hours of the first administered dose, or for multiple, spaced doses throughout the day. The active agents which make up the therapy may be administered simultaneously, either in a combined dosage form or in separate dosage forms intended for substantially simultaneous oral administration. The active agents which make up the therapy may also be administered sequentially, with either active component being administered by a regimen calling for two-step ingestion. Thus, a regimen may call for sequential administration of the active agents with spaced-apart ingestion of the separate, active agents. The time period between the multiple ingestion steps may range from a few minutes to as long as about 72 hours, depending upon the properties of each active agent such a potency, solubility, bioavailability, plasma half-life and kinetic profile of the agent, as well as depending upon the age and condition of the patient. The active agents of the therapy whether administered simultaneously, substantially simultaneously, or sequentially, may involve a regimen calling for administration of one active agent by oral route and the other active agent by intravenous route. Whether the active agents of the therapy are administered by oral or intravenous route, separately or together, each such active agent will be contained in a suitable pharmaceutical formulation of pharmaceutically-acceptable excipients, diluents or other formulations components.

### EXAMPLE 1

This study critically examined the in vivo efficacy of phosphate binding by a combination of phosphate binders, magnesium R-(+)-α-lipoate and calcium succinate monohydrate, in a mouse model of chronic kidney disease (CKD). The model is partial renal ablation in the LDL receptor-deficient (LDLR−/−) mouse that is fed high fat/cholesterol diets. This model resembles the clinical situation of CKD complicated by the metabolic syndrome, because the mice exhibit obesity, hypertension, insulin resistance, and early type II diabetes. In these animals, CKD causes intensification of vascular calcification (VC).

Materials: Calcium succinate monohydrate (CaSucc) and magnesium R-(+)-α-lipoate (MgRALA) having high purity and freedom from contaminating trace metals were provided by BioLink Life Sciences, Inc. Other chemicals were purchased from Sigma Aldrich Company (St. Louis, Mo.).

Animals and Diets: LDL receptor null (LDLR−/−) mice of both genders in a C57BI/6J background were purchased from Jackson Laboratory (Bar Harbor, Me.) and bred in a pathogen-free environment. Animals were weaned at three weeks to a chow diet [1:1 mixture of Pico Lab rodent chow 20 and mouse chow 20, 6.75% calories as fat]. At 10 weeks, animals were continued on this chow diet or initiated on a high cholesterol (0.15%) diet containing 42% calories as fat (Harlan Teklad, Madison Wis., Product No. TD88137), a diet that has been shown to generate atherosclerosis with vascular calcification in mice of this genetic background. Animals had access to water ad libitum, and were maintained according to local and national animal care guidelines. At 12 weeks, CKD was induced as described below.

Induction of CKD and Treatment Protocol: A two-step procedure was utilized to create uremia. Briefly, electrocautery was applied to the right kidney through a 2 cm flank incision at 10 weeks post natal, followed by left total nephrectomy through a similar incision 2 weeks later. Stable CKD was established after the two surgical procedures. Control animals received sham operations in which the appropriate kidney was exposed and mobilized but not treated in any other way.

After the surgical procedures, the 14-week-old mice appearing well and eating were randomized into groups. Group sizes were 10-12 animals in each CKD group and 10 in each sham group. Once the mice were randomized into groups, they were allowed to develop calcification from weeks 14 through weeks 22 post natal. Therapy was initiated at 23 weeks post natal and continued until week 28 weeks post natal at which time the mice were sacrificed.

Two sets of animals in the group receiving magnesium alpha-lipoate were treated according to the protocol. Tissues from the first set of animals were harvested for histologic analysis of calcification and biochemical analysis of aortic calcium. Tissues from the second set of animals in each group were harvested for gene expression in the aorta (real time-RT-PCR) and immunohistochemistry. Myocardin, Smad6, sm22α, calponin, αSMA, BMP-2, RUNX2/Cbfa1, osteopontin and osteocalcin message levels were measured by real time RT-PCR. Osteocalcin, type 1 collagen and αSMA were determined by immunohistochemistry. MGP levels and specifically γ-carboxylated MGP were determined with a specific antibody.

Intraperitoneal anesthesia [xylazine (13 mg/kg) and ketamine (87 mg/kg)] was used for all procedures. Saphenous vein blood samples were taken 1 week following the second surgery to assess baseline post-surgical renal function. Animals were sacrificed under anesthesia 28 weeks post natal. At the time of sacrifice, blood was taken by intracardiac stab, and the heart and aorta dissected en bloc.

Blood Tests: Serum was analyzed on the day of blood draw for blood urea nitrogen [BUN], cholesterol, calcium, glucose and phosphate by standard autoanalyzer laboratory methods. Serum levels of TNF-α, IL-6, and fetuin were measured by immunoassay.

Chemical Calcification Quantitation: Aorta and hearts were dissected at sacrifice, and all extraneous tissue removed by blunt dissection under a dissecting microscope. Tissues were desiccated for 20-24 hours at 55° C., weighed and crushed to a powder with a pestle and mortar. Calcium was eluted into 1 N HCl for 24 hours at 4° C. Calcium content of each eluate was assayed using a cresolphthalein complexone method (Sigma, St Louis), according to the manufacturer's instructions. Results were corrected for dry tissue weight.

Bone Histology and Histomorphometry: Bone formation was determined at the time of sacrifice. All mice received intraperitoneal tetracycline (5 mg/kg) 7 and 2 days before being sacrificed. Both femurs were dissected at the time of sacrifice and placed in 70% ethanol. The specimens were implanted undecalcified in a plastic embedding kit 1-17000 (Energy Beam Sciences, Agawam, Mass.). Bones were sectioned longitudinally through the frontal plane in 5-pm sections with a JB-4 Microtome (Energy Beam Sciences). Tissue was stained with Goldner's trichrome stain for trabecular and cellular analysis. TRAP staining was used to identify osteoclasts and define osteoclast surfaces. Unstained 10-pm sections were used for tetracycline-labeled fluorescence analysis. Slides were examined at 400× magnification using a Leitz microscope attached to an Osteomeasure Image Analyzer (Osteometrics, Atlanta, Ga.). Ten contiguous 0.0225 mm² fields of the distal femur, 150 pm proximal to the growth plate, will be examined per animal. Primary, derived, and kinetic measures of bone remodeling were calculated and reported per guidelines of the American Society of Bone and Mineral Research.

Statistical Analyses: Statistical analyses were performed using ANOVA. Differences between groups were assessed post hoc using Dunnett's multiple range test and considered significant at p<0.05. Data are presented as mean±SE. Analyses were performed using Sigma Stat statistical software (Point Richmond, Calif.).

Summary of Results

Experimental data confirmed that 1% by weight of a combination of calcium succinate and magnesium alpha-lipoate in the diet significantly reduced the uptake of phosphorus from the gastrointestinal tract. Additional effects related to the treatment that were observed in the test groups included beneficial changes in insulin resistance, significant and beneficial changes in serum glucose, and beneficial changes in serum cholesterol. However, adverse effects were also unexpectedly observed in animals receiving this treatment, including hair loss, pustules, and skin disorders. These observations raised concerns that less obvious metabolic dysfunctions (i.e., additional adverse effects not monitored by testing) were also introduced by the treatment. The adverse effects were sufficient to delay further study and development of the test treatment until a solution was obtained.

### EXAMPLE 2

This study critically examined the in vivo efficacy of phosphate binding by a combination of phosphate binders, magnesium R-(+)-α-lipoate and calcium succinate monohydrate, with D-biotin, in a mouse model of chronic kidney disease (CKD). The model is partial renal ablation in the LDL receptor-deficient (LDLR−/−) mouse that is fed high fat/cholesterol diets. This model resembles the clinical situation of CKD complicated by the metabolic syndrome, because the mice exhibit obesity, hypertension, insulin resistance, and early type II diabetes. In these animals, CKD causes intensification of VC.

Materials: Calcium succinate monohydrate (CaSucc) and magnesium R-(+)-α-lipoate (MgRALA) having high purity and freedom from contaminating trace metals were provided by BioLink Life Sciences, Inc. Other chemicals, including D-biotin, were purchased from Sigma Aldrich Company (St. Louis, Mo.).

Animals and Diets: LDL receptor null (LDLR−/−) mice of both genders in a C57BI/6J background were purchased from Jackson Laboratory (Bar Harbor, Me.) and bred in a pathogen-free environment. Animals were weaned at three weeks to a chow diet [1:1 mixture of Pico Lab rodent chow 20 and mouse chow 20, 6.75% calories as fat]. At 10 weeks, animals were continued on this chow diet or initiated on a high cholesterol (0.15%) diet containing 42% calories as fat (Harlan Teklad, Madison Wis., Product No. TD88137), a diet that has been shown to generate atherosclerosis with vascular calcification in mice of this genetic background. Animals had access to water ad libitum, and were maintained according to local and national animal care guidelines. At 12 weeks, CKD was induced as described below. Induction of CKD and Treatment Protocol: A two-step procedure was utilized to create uremia. Briefly, electrocautery was applied to the right kidney through a 2 cm flank incision at 10 weeks post natal, followed by left total nephrectomy through a similar incision 2 weeks later. Stable CKD was established after the two surgical procedures. Control animals received sham operations in which the appropriate kidney was exposed and mobilized but not treated in any other way.

After the surgical procedures, the 14-week-old mice appearing well and eating were randomized into groups. Group sizes were 10-12 animals in each CKD group and 10 in each sham group. Once the mice were randomized into groups, they were allowed to develop calcification from weeks 14 through weeks 22 post natal. Therapy was initiated at 23 weeks post natal and continued until week 28 weeks post natal at which time the mice were sacrificed.

Two sets of animals in the test group receiving magnesium alpha-lipoate were treated according to the protocol. Tissues from the first set of animals were harvested for histologic analysis of calcification and biochemical analysis of aortic calcium. Tissues from the second set of animals in each group were harvested for gene expression in the aorta (real time-RT-PCR) and immunohistochemistry. Myocardin, Smad6, sm22α, calponin, αSMA, BMP-2, RUNX2/Cbfa1, osteopontin and osteocalcin message levels were measured by real time RT-PCR. Osteocalcin, type 1 collagen and αSMA were determined by immunohistochemistry. MGP levels and specifically γ-carboxylated MGP were determined with a specific antibody.

Intraperitoneal anesthesia [xylazine (13 mg/kg) and ketamine (87 mg/kg)] was used for all procedures. Saphenous vein blood samples were taken 1 week following the second surgery to assess baseline post-surgical renal function. Animals were sacrificed under anesthesia 28 weeks post natal. At the time of sacrifice, blood was taken by intracardiac stab, and the heart and aorta dissected en bloc.

Blood Tests: Serum was analyzed on the day of blood draw for blood urea nitrogen [BUN], cholesterol, calcium, glucose and phosphate by standard autoanalyzer laboratory methods. Serum levels of TNF-α, IL-6, and fetuin were measured by immunoassay.

Chemical Calcification Quantitation: Aorta and hearts were dissected at sacrifice, and all extraneous tissue removed by blunt dissection under a dissecting microscope. Tissues were desiccated for 20-24 hours at 55° C., weighed and crushed to a powder with a pestle and mortar. Calcium was eluted into 1 N HCl for 24 hours at 4° C. Calcium content of each eluate was assayed using a cresolphthalein complexone method (Sigma, St Louis), according to the manufacturer's instructions. Results were corrected for dry tissue weight.

Bone Histology and Histomorphometry: Bone formation was determined at the time of sacrifice. All mice received intraperitoneal tetracycline (5 mg/kg) 7 and 2 days before being sacrificed. Both femurs were dissected at the time of sacrifice and placed in 70% ethanol. The specimens were implanted undecalcified in a plastic embedding kit 1-17000 (Energy Beam Sciences, Agawam, Mass.). Bones were sectioned longitudinally through the frontal plane in 5-pm sections with a JB-4 Microtome (Energy Beam Sciences). Tissue was stained with Goldner's trichrome stain for trabecular and cellular analysis. TRAP staining was used to identify osteoclasts and define osteoclast surfaces. Unstained 10-pm sections were used for tetracycline-labeled fluorescence analysis. Slides were examined at 400× magnification using a Leitz microscope attached to an Osteomeasure Image Analyzer (Osteometrics, Atlanta, Ga.). Ten contiguous 0.0225 mm² fields of the distal femur, 150 pm proximal to the growth plate, will be examined per animal. Primary, derived, and kinetic measures of bone remodeling were calculated and reported per guidelines of the American Society of Bone and Mineral Research.

Statistical Analyses: Statistical analyses were performed using ANOVA. Differences between groups were assessed post hoc using Dunnett's multiple range test and considered significant at p<0.05. Data are presented as mean±SE. Analyses were performed using Sigma Stat statistical software (Point Richmond, Calif.).

Summary of Results

Experimental data confirmed that 1% by weight of a combination of calcium succinate and magnesium alpha-lipoate with 10 mg D-biotin in the diet significantly reduced the uptake of phosphorus from the gastrointestinal tract. Additional effects related to the treatment that were observed in the test groups included beneficial changes in insulin resistance, significant beneficial changes in serum glucose, and beneficial changes in serum cholesterol. The incidences of hair loss, pustules, and skin disorders were significantly reduced or eliminated. No adverse effects were introduced by the treatment.

All mentioned references are incorporated by reference as if here written. When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

Without further elaboration, it is believed that one skilled in the art can, using the preceding description, utilize the present invention to its fullest extent. The specific embodiments are, therefore, to be construed as merely illustrative, and not limitative of the remainder of the disclosure in any way whatsoever.

I claim:

1. A composition for enhancing the safety of and reducing the adverse effects related to administration of magnesium alpha-lipoate, comprising magnesium alpha-lipoate with D-biotin, wherein the dosage of magnesium alpha-lipoate in the composition comprises about 0.1 to about 100 milligrams magnesium, on an elemental basis, in the form of magnesium alpha-lipoate and the dosage of D-biotin in the composition comprises about 0.01 to about 50 milligrams D-biotin.

2. A method of enhancing the safety of and reducing the adverse effects caused by a pharmaceutical composition comprising magnesium alpha-lipoate when administered to a warm-blooded animal, comprising administering to said warm-blooded animal in a coordinated manner a first dosage of magnesium alpha-lipoate and a second dosage of D-biotin, wherein said first dosage comprises about 0.1 to about 100 milligrams magnesium, on an elemental basis, in the form of magnesium alpha-lipoate and said second dosage comprises about 0.01 to about 50 milligrams D-biotin.

3. An oral composition useful for enhancing the safety of and reducing the adverse effects caused by magnesium alpha-lipoate following administration to a warm-blood animal, comprising a unit dosage or serving of from about 5 milligrams to about 100 milligrams magnesium, on an elemental basis, in the form of magnesium alpha-lipoate and from about 0.01 milligrams to about 50 milligrams D-biotin.

4. A method of enhancing the safety of and reducing the adverse effects caused by magnesium R-(+)-alpha-lipoate following administration to a human, comprising administering to said human a first dosage of magnesium R-(+)-alpha-lipoate and a second dosage of D-biotin, wherein said first dosage comprises about 0.1 to about 100 milligrams magnesium, on an elemental basis, in the form of magnesium R-(+)-alpha-lipoate and said second dosage comprises about 0.01 to about 50 milligrams D-biotin.

5. The composition of claim 1, wherein the dosage of magnesium alpha-lipoate in the composition comprises about 0.1 to about 100 milligrams magnesium, on an elemental basis, in the form of magnesium alpha-lipoate, administered by mouth, and the dosage of D-biotin in the composition comprises about 0.01 to about 50 milligrams D-biotin, administered orally or intravenously, within about 72 hours of administration of the magnesium alpha-lipoate.

6. The method of claim 2, wherein administering to said mammal in a coordinated manner a first dosage of magnesium alpha-lipoate and a second dosage of D-biotin, wherein said first dosage comprises about 0.1 to about 100 milligrams magnesium, on an elemental basis, in the form of magnesium alpha-lipoate and said second dosage comprises about 0.01 to about 50 milligrams D-biotin further comprises administering of said second dosage within 72 hours of administering said first dosage.
